(19) United States
(12) Patent Application Publication
(10) Pub. No.: US 2015/0164379 A1
(43) Pub. Date: Jun. 18, 2015

(54) METHOD AND APPARATUS FOR DETECTING AND/OR ANALYZING MOTION USING RADAR AND MULTIPLE IDENTIFIABLE REFLECTORS

(71) Applicant: University of Florida Research Foundation, Incorporated, Gainesville, FL (US)

(72) Inventors: Jenshan Lin, Gainesville, FL (US); Nikolaus Gravenstein, Gainesville, FL (US)

(73) Assignee: University of Florida Research Foundation, Incorporated, Gainesville, FL (US)

(21) Appl. No.: 14/530,711

(22) PCT Filed: May 23, 2013
(86) PCT No.: PCT/US2013/042476

§ 371 Date: Nov. 1, 2014

Related U.S. Application Data
(60) Provisional application No. 61/650,718, filed on May 23, 2012.

(51) Int. Cl. A61B 5/11; A61B 5/0205; A61B 5/00; A61B 19/00; A61B 5/113

(52) U.S. Cl. CPC A61B 5/1127; A61B 19/54; A61B 5/113; A61B 19/44; A61B 5/7278; A61B 5/0205; A61B 2019/547; A61B 2019/448; A61B 2505/07; A61B 2503/40; A61B 5/0826

(57) ABSTRACT

Embodiments of the subject invention relate to a method and apparatus for detecting and/or analyzing respiratory motion of a patient, such as an animal or person. A specific embodiment can use a radar motion detector to measure and record respiratory movements at different parts of the body, using multiple identifiable markers, such as reflectors, positioned such that movements of the patient can be monitored by monitoring the movement of the markers. As an example, the markers can be attached to or worn by the patient, inserted into the patient (such as under the skin), or otherwise positioned in a known relation to a portion or surface of the patient. In an embodiment, the identifiable reflectors are transponders with different modulation codes (in frequency, phase, amplitude, or combinations thereof) on the reflected signal. A modulation frequency higher than the respiration rate can be used so that the multiple reflected signals received from the reflectors can be separated and respiration signal modulating on the reflected radio frequency signal from each reflector can be extracted and analyzed.

### BACKGROUND

While breathing, a person has respiratory movements on different parts of the body. These respiratory movements may have different amplitudes and/or different phases/delays. Detecting and/or analyzing these movements may help diagnose a patient's condition or identify potential health problems of the patient. As an example, obstructive breathing can result in paradox respiratory movements at chest and abdomen, such that detection and/or analysis of such paradox respiratory movements of the chest and abdomen can be used as an indication of obstructive breathing.

### BRIEF SUMMARY

Embodiments of the subject invention relate to a method and apparatus for detecting and/or analyzing motion of a patient, such as an animal or person. Specific embodiments relate to a method and apparatus for detecting and/or analyzing respiratory motion of a patient, such as an animal or person. A specific embodiment can use a radar motion detector to measure and record respiratory movements at different parts of the body, using multiple identifiable markers, such as reflectors, positioned such that movements of the patient can be monitored by monitoring the movement of the markers. As an example, the markers can be attached to or worn by the patient, inserted into the patient (such as under the skin), or otherwise positioned in a known relation to a portion or surface of the patient. Specific embodiments can utilize transponders, such as RF transponders, as identifiable reflectors, where the transponders emit an identifying signal in response to an interrogating received signal. In an embodiment, the identifiable reflectors are transponders with different modulation codes (in frequency, phase, amplitude, or combinations thereof) on the reflected signal. A modulation frequency higher than the respiration rate can be used so that the multiple reflected signals received from the reflectors can be separated and respiration signal modulating on the reflected radio frequency signal from each reflector can be extracted and analyzed.

Further specific embodiments can use a radar motion detector to measure and record movements, such as cardiac movements, of a patient, using at least one identifiable marker, such as reflector(s), positioned such that movements of the patient can be monitored by monitoring the movement of the markers.

Specific embodiments can determine whether the patient has obstructive breathing, determine different amplitudes and/or different phases/delays, diagnose a patient's condition or health problems. In particular, paradox respiratory movements of the chest and/or abdomen, such as the chest expanding when the abdomen contracts and/or the abdomen expanding when the chest contracts, can be identified and used as an indication of obstructive breathing.

### BRIEF DESCRIPTION OF DRAWINGS

FIG. 1 shows a system for Doppler radar movement monitoring using harmonic tags in accordance with an embodiment of the subject invention.

FIG. 2 shows an embodiment of an RF tag used as an on-body sensor (not to scale).

### DETAILED DISCLOSURE

Embodiments of the subject invention relate to a method and apparatus for detecting and/or analyzing motion of a patient, such as an animal or person. Specific embodiments relate to a method and apparatus for detecting and/or analyzing respiratory motion of a patient, such as an animal or person. A specific embodiment can use a radar motion detector to measure and record respiratory movements at different parts of the body, using multiple identifiable markers, such as reflectors, positioned such that movements of the patient can be monitored by monitoring the movement of the markers. As an example, the markers can be attached to or worn by the patient, inserted into the patient (such as under the skin), or otherwise positioned in a known relation to a portion or surface of the patient. Specific embodiments can utilize transponders, such as RF transponders, as identifiable reflectors, where the transponders emit an identifying signal in response to an interrogating received signal. In an embodiment, the identifiable reflectors are transponders with different modulation codes (in frequency, phase, amplitude, or combinations thereof) on the reflected signal. A modulation frequency higher than the respiration rate can be used so that the multiple reflected signals received from the reflectors can be separated and respiration signal modulating on the reflected radio frequency signal from each reflector can be extracted and analyzed.

Further specific embodiments can use a radar motion detector to measure and record movements, such as cardiac movements, of a patient, using at least one identifiable marker, such as reflector(s), positioned such that movements of the patient can be monitored by monitoring the movement of the markers.

Embodiments can be utilized as respiration monitoring devices and systems in hospitals, sleep test labs, or even patients' homes. Embodiments can replace existing devices and systems in order to improve the accuracy of the respiration measurements. The reflectors can be made very small and lightweight, such that patients will feel more comfortable when having their respiration measured. Specific embodiments can be non-contact with the exception of the reflectors, such that the reflectors are the only parts of the system or operator that need to make contact with the patient. In specific embodiments, at least one marker, such as a reflector, is used in order to optimize identification of the motion source and determination of the motion being monitored. In further embodiments, at least two markers, at least 3 markers, at least 4 markers, and at least 5 markers are used. Specific embodiments place the receiver a distance from the markers and/or the markers from the transmit antenna, in the range of 1-2 meters, 2-3 meters, 3-4 meters, and/or 4-5 meters.

Radio frequency identification (RFID) tags can be used as reflectors for specific embodiments, such that the RFID tags are attached to the patient, or positioned with respect to the patient, such that the RFID tags move as the patient breathes and follow the motion of the patient. As an example, the reflectors can be attached or positioned on the patient's clothing or other object or device having a known physical relationship with one or more locations on the patient. In a specific embodiment, one or more of the reflectors can be inserted under the patient's skin. The RF signal can then be targeted onto the tags and the reflected and/or return RF signal received and demodulated to detect the patient motion.

With respect to a specific embodiment for determining a patient's breathing motion, a first marker can be positioned on the chest at a point of chest wall movement, and preferably at a point of significant chest wall movement, and a second marker can be positioned on the abdomen, for a total of two markers. Of course, additional markers can be used for additional accuracy and/or additional information. An embodiment that can be useful for heart rate diagnosis can position a marker on the chest, and a second marker on the neck, head, or other position that provides a strong cardiac pulsation. Again, one or more additional markers can be used to provide additional accuracy and/or additional information. Further, when indicating the marker is on the chest, on the abdomen, on the neck, or on the head, the marker can also be positioned relative to the chest, abdomen, neck, or head in such a way that the marker experiences a motion that tracks the motion of the chest, abdomen, neck, or head, respectively. Specific embodiments can determine whether the chest and abdomen are in sync or out of sync with each other during breathing and, if out of sync, by how much.

A variety of techniques can be used to create the RF signal transmitted to the reflectors, receive the reflected and/or return RF signal, and process the reflected and/or return RF signal. In various embodiments, an RFID tag/RFID system that can modulate ID code onto the reflected carrier, and the receiver can receive the reflected wave/signal can identify which marker, such as a reflector, reflected the particular signal back to the receiver can be utilized. Systems using, for example, passive RFID tags and/or active transponder RFID tags can be used. In a specific embodiment, a Doppler radar system as taught in “Respiratory Monitoring and Clutter Rejection using a CW Doppler Radar with Passive RF Tags”, IEEE Sensors Journal, March 2012, Vol. 12, No. 3, pp. 558-565, which is hereby incorporated by reference in its entirety, can be used to monitor the motion of one or more reflectors positioned relative to a corresponding one or more locations of a patient's body, in order to monitor one or more aspects of the patient's breathing.

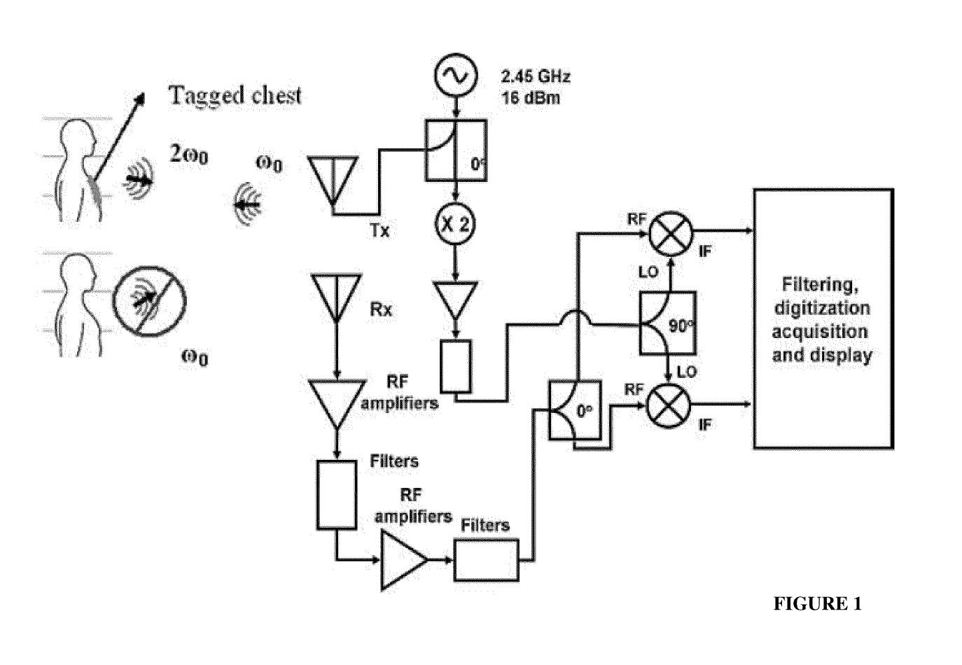

FIG. 1 shows a system for Doppler radar movement monitoring using harmonic tags in accordance with an embodiment of the subject invention. The base-band output after mixing the received and local oscillator signals is given by

$${I}_{BB} = A cos [ \frac{4 π f}{c} x ( t ) + {ϕ}_{tot} ] \qquad ( 1 )$$

where, x(t)=h(t)+r(t), is the chest motion composed of heart motion (h(t)) and the respiration (r(t)). φₜₒₜ is the residual phase noise in the system. A radio frequency wave is incident on the tag, and the periodic motion of the tag is converted into a phase shift in the reflected signal. The phase shift is directly proportional to the movement of the tag. This phase shift is detected and processed to determine the movement of the tag.

When a harmonic tag is placed on human body; in the simplest case, ignoring the phase noise of the oscillator and the phase shift due to the distance of the target, the signal at the receiving antenna will consist mainly of two components: leakage at 2.45 GHz and an rf signal from tag reflection at 4.9 GHz. These signals could be represented as:

$${A}_{rf} cos [ ω t - \frac{2 ω d}{c} - \frac{2 ω x ( t )}{c} ] + {A}_{rh} cos [ 2 ω t - \frac{4 ω d}{c} - \frac{4 ω x ( t )}{c} ] \qquad ( 2 )$$

Where the term cot represents the fundamental frequency of 2.45 GHz, d represents the nominal distance between the transmitting antenna and x(t) is the periodic motion of the target. The terms A_{rf} and Aᵣₕ represent the amplitude variations corresponding to the received fundamental and harmonic components of signal respectively. The LO signal without the phase noise can be represented as

A_{L} cos(2ωt)

After mixing, the required baseband signal

$${A}_{L} {A}_{rf} cos = [ \frac{4 ω d}{c} + \frac{4 ω x ( t )}{c} ] \qquad ( 4 )$$

is filtered out and decoded to yield the respiration rate.

A quadrature receiver is used to alleviate measurement issues with null points occurring every lambda/4 distance. On the complex I-Q plot, these equations form an arc that belongs to a circle centered at the origin.

For efficient operation, the antenna tag in RFID is preferably designed to present an appropriate impedance match to the RFID chip. In a wearable tag, the human body presents a large conducting mass in close proximity, and is thus an integral part of the antenna design. The effect is detrimental, in that the body blocks and absorbs RF energy, and complicates impedance matching in a variable manner that is difficult to quantify.

In a specific embodiment, a harmonic tag can be used that relies on a Doppler shift in a 4.9 GHz harmonic backscatter signal. A specific embodiment uses a planar tag design that allows the operation of the tag at lower powers and has a match between the diode and tag element. The tag can have an impedance somewhat close to being the conjugate of the diode reactance at both 2.45 GHz and 4.9 GHz. The tag can be constructed with a copper tape. The gain of a specific tag antenna at 2.45 GHz is 5 dB and at 4.9 GHz is 5.2 dB.

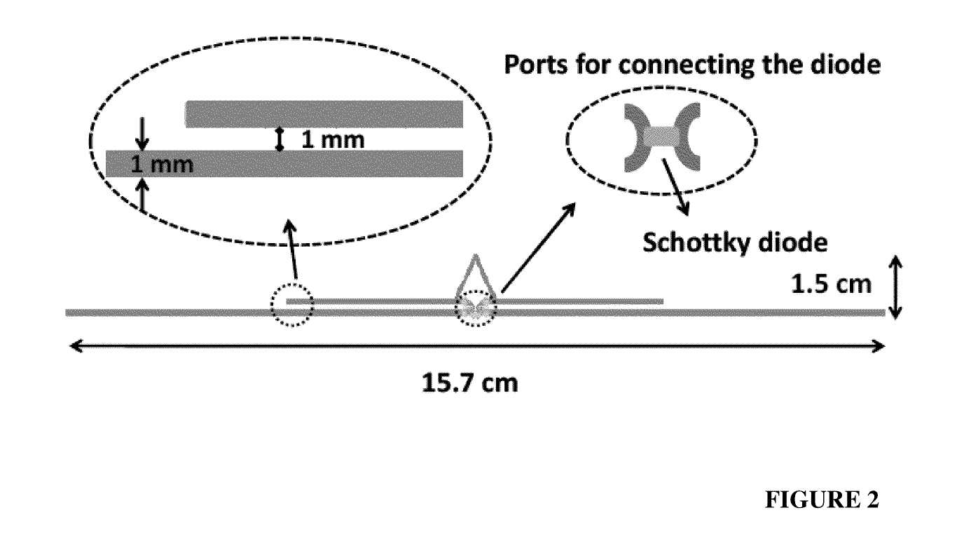

The tag can be placed over a material, such as a 0.5 cm Styrofoam substrate, in order to minimize the effect of the human body on the EM field of the antenna. A specific embodiment of such a tag, along with its dimensions, is shown in FIG. 2. For receiving the tag signal at 4.9 GHz, an array of micro-strip antenna having a gain of 5.82 dB can be used.

In an embodiment, a transmitted power of 10 dBm at 2.45 GHz can be used. The tags can be placed at a distance in the range of 0.1 m-0.5 m, 0.2 m-0.4 m, 0.5 m-1 m, in the range 0.6 m-0.9 m, and/or in the range 0.7 m-0.8 m.

Aspects of the invention, such as transmission of the RF signal, receipt of the reflected and/or return RF signal, and processing of the reflected and/or return RF signal, may be described in the general context of computer-executable instructions, such as program modules, being executed by a computer. Generally, program modules include routines, programs, objects, components, data structures, etc., that perform particular tasks or implement particular abstract data types. Moreover, those skilled in the art will appreciate that the invention may be practiced with a variety of computer-system configurations, including multiprocessor systems, microprocessor-based or programmable-consumer electronics, minicomputers, mainframe computers, and the like. Any number of computer-systems and computer networks are acceptable for use with the present invention.

Specific hardware devices, programming languages, components, processes, protocols, and numerous details including operating environments and the like are set forth to provide a thorough understanding of the present invention. In other instances, structures, devices, and processes are shown in block-diagram form, rather than in detail, to avoid obscuring the present invention. But an ordinary-skilled artisan would understand that the present invention may be practiced without these specific details. Computer systems, servers, work stations, and other machines may be connected to one another across a communication medium including, for example, a network or networks.

As one skilled in the art will appreciate, embodiments of the present invention may be embodied as, among other things: a method, system, or computer-program product. Accordingly, the embodiments may take the form of a hardware embodiment, a software embodiment, or an embodiment combining software and hardware. In an embodiment, the present invention takes the form of a computer-program product that includes computer-useable instructions embodied on one or more computer-readable media.

Computer-readable media include both volatile and nonvolatile media, transient and non-transient, removable and nonremovable media, and contemplate media readable by a database, a switch, and various other network devices. By way of example, and not limitation, computer-readable media comprise media implemented in any method or technology for storing information. Examples of stored information include computer-useable instructions, data structures, program modules, and other data representations. Media examples include, but are not limited to, information-delivery media, RAM, ROM, EEPROM, flash memory or other memory technology, CD-ROM, digital versatile discs (DVD), holographic media or other optical disc storage, magnetic cassettes, magnetic tape, magnetic disk storage, and other magnetic storage devices. These technologies can store data momentarily, temporarily, or permanently.

The invention may be practiced in distributed-computing environments where tasks are performed by remote-processing devices that are linked through a communications network. In a distributed-computing environment, program modules may be located in both local and remote computer-storage media including memory storage devices. The computer-useable instructions form an interface to allow a computer to react according to a source of input. The instructions cooperate with other code segments to initiate a variety of tasks in response to data received in conjunction with the source of the received data.

The present invention may be practiced in a network environment such as a communications network. Such networks are widely used to connect various types of network elements, such as routers, servers, gateways, and so forth. Further, the invention may be practiced in a multi-network environment having various, connected public and/or private networks.

Communication between network elements may be wireless or wireline (wired). As will be appreciated by those skilled in the art, communication networks may take several different forms and may use several different communication protocols. And the present invention is not limited by the forms and communication protocols described herein.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A method for monitoring a patient's movements, comprising:
positioning at least two markers in a corresponding at least two relative positions with respect to a corresponding at least two locations of a patient's body;
transmitting one or more transmit RF signal at the at least two markers, wherein the at least two markers cause a corresponding at least two return RF signals due to the one or more transmit RF signal;
receiving the at least two return RF signals; and
processing the at least two return RF signals to determine information regarding a corresponding at least two movements of the at least two locations of the patient's body.

2. The method according to claim 1, further comprising:
determining information regarding the patient's breathing from the information regarding the at least two movements.

3. The method according to claim 2, wherein a first location of the at least two locations of the patient's body is proximate a chest wall of the patient.

4. The method according to claim 3, wherein a second location of the at least two locations of the patient's body is proximate an abdomen of the patient.

5. The method according to claim 2, wherein the information regarding the patient's breathing includes information regarding whether the patient has obstructive breathing.

6. The method according to claim 2, wherein the information regarding the patient's breathing includes a difference in amplitude of two of the at least two movements.

7. The method according to claim 2, wherein the information regarding the patient's breathing includes a difference in phase of two of the at least two movements.

8. The method according to claim 5, wherein the information regarding the patient's breathing further includes a difference in phase of the two of the at least two movements.

9. The method according to claim 4, wherein the information regarding the patient's breathing includes a difference in amplitude of a movement of the chest wall and a movement of the abdomen.

10. The method according to claim 4, wherein the information regarding the patient's breathing includes a difference in phase of a movement of the chest wall and a movement of the abdomen.

11. The method according to claim 9, wherein the information regarding the patient's breathing includes a difference in phase of the movement of the chest wall and the movement of the abdomen.

12. The method according to claim 1, wherein the at least two markers are at least two RFID tags.

13. The method according to claim 1, wherein the at least two markers are at least two harmonic RFID tags.

14. The method according to claim 13, wherein the at least two return RF signals are each a harmonic of one of the one or more transmit RF signal.

15. The method according to claim 13, wherein the at least two return RF signals are a second harmonic of one of the one or more transmit RF signal.

16. The method according to claim 1, wherein one or more of the at least two markers are attached to the patient's body.

17. The method according to claim 1, wherein one or more of the at least two markers are positioned in a known relation to the patient.

18. The method according to claim 1, wherein one or more of the at least two markers are inserted into the patient.

19. The method according to claim 1, wherein the information regarding the patient's breathing is information regarding paradox respiratory movements.

20. The method according to claim 1, wherein at least one of the at least two return RF signals provides information regarding an identity of the patient.

21. The method according to claim 1, wherein at least one of the at least two return RF signals provides an identifying code.

22. The method according to claim 1, wherein transmitting the one or more transmit RF signal comprises transmitting the one or more transmit RF signal via a corresponding one or more transmitter having a corresponding one or more transmit antenna.

23. The method according to claim 21, wherein a distance between at least one of the one or more transmit antenna and at least one of the at least two markers is in the range of 1-2 m.

24. The method according to claim 21, wherein a distance between at least one of the one or more transmit antenna and at least one of the at least two markers is in the range of 2-3 m.

25. The method according to claim 21, wherein a distance between at least one of the one or more transmit antenna and at least one of the at least two markers is in the range of 3-4 m.

26. The method according to claim 21, wherein a distance between at least one of the one or more transmit antenna and at least one of the at least two markers is in the range of 4-5 m.

27. The method according to claim 21, wherein a distance between at least one of the one or more transmit antenna and at least one of the at least two markers is in the range of 0.1-1.0 m.

28. The method according to claim 1, wherein the patient is a human.

29. The method according to claim 1, wherein the patient is an animal.

30. The method according to claim 1, wherein the one or more transmit RF signal is a single transmit RF signal.

31. A method for monitoring a patient's movements, comprising:
positioning at least one marker in a corresponding at least one relative position with respect to a corresponding at least one location of a patient's body;
transmitting one or more transmit RF signal at the at least one marker, wherein the at least one marker causes a corresponding at least one return RF signal due to the one or more transmit RF signal;
receiving the at least one return RF signal; and
processing the at least one return RF signal to determine information regarding a corresponding at least one movement of the at least one location of the patient's body; and determining information regarding the patient's cardiac functioning from the information regarding at least one movement.

32. The method according to claim 30, wherein the information regarding the patient's cardiac functioning comprises the patient's heart rate.

33. The method according to claim 31, wherein the at least one marker comprises at least two markers, wherein the at least one relative position comprises at least two relative positions, wherein the at least one location of a patient's body comprises at least two locations of a patient's body, wherein the at least one return RF signal comprises at least two return RF signals, wherein the at least one movement comprises at least two movements.

34. The method according to claim 29, wherein a first location of the at least two locations of the patient's body is proximate a chest of the patient, wherein a second location of the at least two locations of the patient's body is proximate a head of the patient.

35. The method according to claim 29, wherein a first location of the at least two locations of the patient's body is proximate a chest of the patient, wherein a second location of the at least two locations of the patient's body is proximate a neck of the patient.

36. The method according to claim 30, wherein the at least one marker is a single marker.

37. The method according to claim 36, wherein the at least one location of the patient's body is a single location of the patient's body, wherein the location of the patient's body is proximate a chest of the patient.

38. The method according to claim 36, wherein the at least one location of the patient's body is a single location of the patient's body, wherein the location of the patient's body is proximate a neck of the patient.

39. A system for monitoring a patient's movements, comprising:
at least two markers, wherein the at least two markers are configured to be positioned in a corresponding at least two relative positions with respect to a corresponding at least two locations of a patient's body;
one or more transmitter, wherein the one or more transmitter transmits one or more transmit RF signal at the at least two markers, wherein the at least two markers cause a corresponding at least two return RF signals due to the one or more transmit RF signal;
at least one receiver, wherein the at least one receiver receives the at least two return RF signals; and
a processor, wherein the processor processes the at least two return RF signals to determine information regarding a corresponding at least two movements of the at least two locations of the patient's body.

40. A system for monitoring a patient's movements, comprising:
at least one marker, wherein the at least one marker is configured to be positioned in a corresponding at least one relative position with respect to a corresponding at least one location of a patient's body;
one or more transmitter, wherein the one or more transmitter transmits one or more transmit RF signal at the at least one marker, wherein the at least one marker causes a corresponding at least one return RF signal due to the one or more transmit RF signal;
at least one receiver, wherein the at least one receiver receives the at least one return RF signal; and
a processor, wherein the processor processes the at least one return RF signal to determine information regarding a corresponding at least one movement of the at least one location of the patient's body; and determining information regarding the patient's cardiac functioning from the information regarding at least one movement.

41. A non-transitory media storage device having machine-readable instructions stored thereon for performing a method for monitoring a patient's movements, the method comprising:
positioning at least two markers in a corresponding at least two relative positions with respect to a corresponding at least two locations of a patient's body;
transmitting one or more transmit RF signal at the at least two markers, wherein the at least two markers cause a corresponding at least two return RF signals due to the one or more transmit RF signal;
receiving the at least two return RF signals; and
processing the at least two return RF signals to determine information regarding a corresponding at least two movements of the at least two locations of the patient's body.

42. A non-transitory media storage device having machine-readable instructions stored thereon for performing a method for monitoring a patient's movements, the method comprising:
positioning at least one marker in a corresponding at least one relative position with respect to a corresponding at least one location of a patient's body;
transmitting one or more transmit RF signal at the at least one marker, wherein the at least one marker causes a corresponding at least one return RF signal due to the one or more transmit RF signal;
receiving the at least one return RF signal; and
processing the at least one return RF signal to determine information regarding a corresponding at least one movement of the at least one location of the patient's body; and determining information regarding the patient's cardiac functioning from the information regarding at least one movement.
